(19) United States
(12) Patent Application Publication
(10) Pub. No.: US 2023/0091212 A1
(43) Pub. Date: Mar. 23, 2023

(54) Phlebotomy Cuff Assembly

(71) Applicant: Ishmael Mayor, North Salt Lake City, UT (US)

(72) Inventor: Ishmael Mayor, North Salt Lake City, UT (US)

(21) Appl. No.: 17/476,022

(22) Filed: Sep. 15, 2021

(51) Int. Cl. A61B 17/135

(52) U.S. Cl. CPC A61B 17/135; A61B 5/15003

(57) ABSTRACT

A phlebotomy cuff assembly for enhancing visibility of veins for phlebotomy includes a cuff that can be wrapped around a patient's limb and a bladder is integrated into the cuff. A first mating member is coupled to the cuff, a second mating member is coupled to the cuff, and the second mating member is matable to the first mating member for forming the cuff into a closed loop around the patient's limb. A hose is fluidly coupled to the bladder and a bulb is fluidly coupled to the hose such that the bulb can be compressed by a caregiver for inflating the bladder. In this way the bladder can compress against the patient's limb to make veins in the patient's limb more visible.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to cuff devices and more particularly pertains to a new cuff device for enhancing visibility of veins for phlebotomy. The device includes a cuff, a bladder integrated into the cuff and a bulb in fluid communication with the bladder. The cuff is positioned around a patient's arm and the bulb is compressed to inflate the bladder. In this way the bladder compresses against the patient's arm to enhance visibility of veins for performing phlebotomy.

### (2) DESCRIPTION OF RELATED ART INCLUDING INFORMATION DISCLOSED UNDER 37 CFR 1.97 AND 1.98

The prior art relates to cuff devices including a variety of electronic tourniquets in communication with a display unit for checking blood pressure. The prior art discloses an inflatable band that is positionable around a patient's limb for forming a tourniquet. The prior art discloses a modular wound treatment device that includes a cuff and a variety of pneumatically actuated components that are attachable to the cuff.

### BRIEF SUMMARY OF THE INVENTION

An embodiment of the disclosure meets the needs presented above by generally comprising a cuff that can be wrapped around a patient's limb and a bladder is integrated into the cuff. A first mating member is coupled to the cuff, a second mating member is coupled to the cuff, and the second mating member is matable to the first mating member for forming the cuff into a closed loop around the patient's limb. A hose is fluidly coupled to the bladder and a bulb is fluidly coupled to the hose such that the bulb can be compressed by a caregiver for inflating the bladder. In this way the bladder can compress against the patient's limb to make veins in the patient's limb more visible.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a phlebotomy cuff assembly according to an embodiment of the disclosure.

FIG. 2 is a front phantom view of an embodiment of the disclosure.

FIG. 3 is a back phantom view of an embodiment of the disclosure.

FIG. 4 is a perspective in-use view of an embodiment of the disclosure.

### DETAILED DESCRIPTION OF THE INVENTION

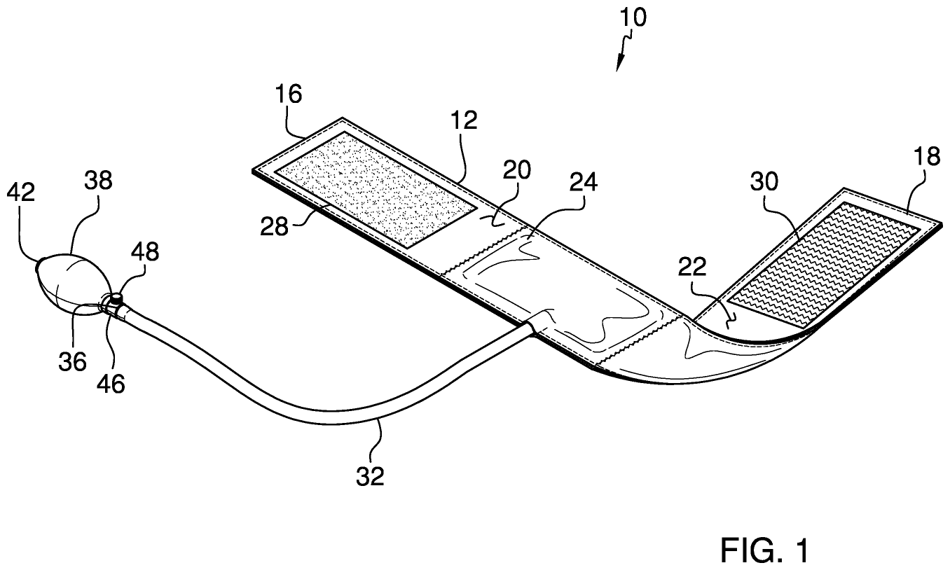

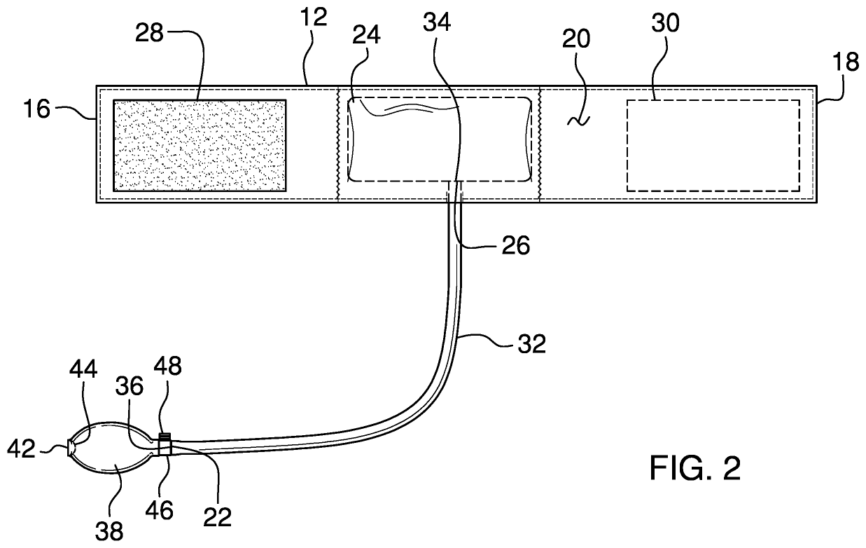

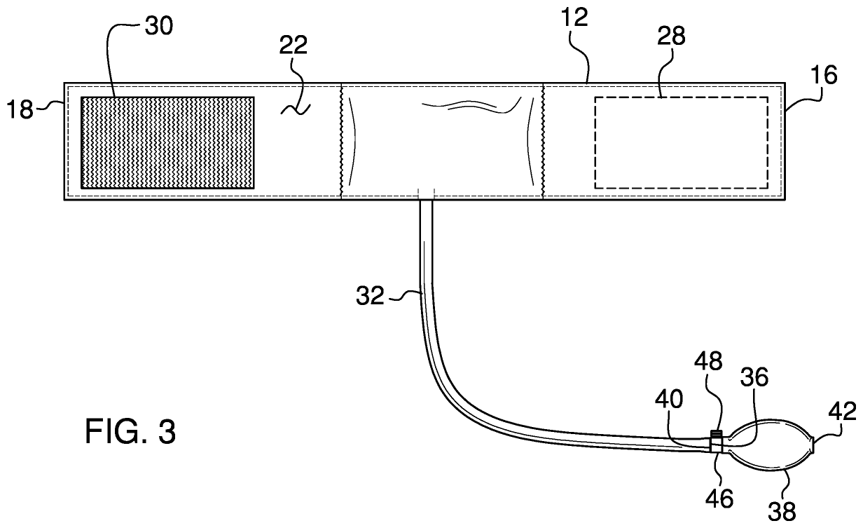

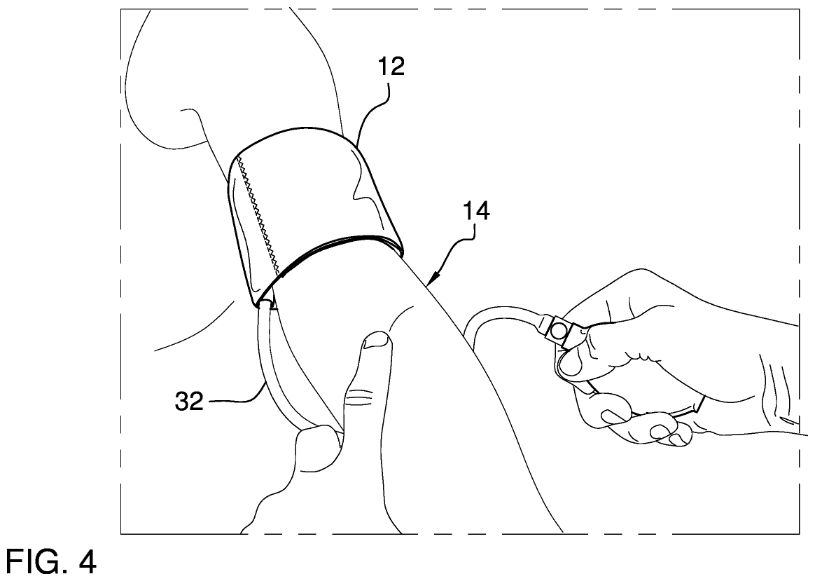

With reference now to the drawings, and in particular to FIGS. 1 through 4 thereof, a new cuff device embodying the principles and concepts of an embodiment of the disclosure and generally designated by the reference numeral 10 will be described.

As best illustrated in FIGS. 1 through 4, the phlebotomy cuff assembly 10 generally comprises a cuff 12 that can be wrapped around a patient's limb 14. The cuff 12 has a first end 16, a second end 18, a first surface 20 extending between the first end 16 and the second end 18, and a second surface 22 extending between the first end 16 and the second end 18. The cuff 12 is elongated between the first end 16 and the second end 18 to a length that is sufficient to facilitate the cuff 12 to be wrapped around the patient's upper arm, for example. A bladder 24 is integrated into the cuff 12 and the bladder 24 is positioned between the first surface 20 and the second surface 22. The bladder 24 is centrally positioned between the first end 16 and the second end 18. Furthermore, the bladder 24 is comprised of a fluid impermeable material such that the bladder 24 can be inflated, and the bladder 24 has an inlet 26.

A first mating member 28 is provided and the first mating member 28 is coupled to the cuff 12. The first mating member 28 is positioned on the first surface 20 and the first mating member 28 is positioned adjacent to the first end 16. A second mating member 30 is provided and the second mating member 30 is coupled to the cuff 12. The second mating member 30 is matable to the first mating member 28 for forming the cuff 12 into a closed loop. In this way the cuff 12 can be retained around the patient's limb 14. The second mating member 30 is positioned on the second surface 22 and the second mating member 30 is positioned adjacent to the second end 18. Each of the first mating member 28 and the second mating member 30 may comprise complementary portions of a hook and loop fastener or other type of releasable, multiple use fastener.

A hose 32 is fluidly coupled to the bladder 24 and the hose 32 extends outwardly from the cuff 12. The hose 32 has a first end 34 and a second end 36, and the first end 34 of the hose 32 is fluidly coupled to the inlet 26 on the bladder 24. A bulb 38 is fluidly coupled to the hose 32 such that the bulb 38 can be compressed by a caregiver. The bulb 38 urges air inwardly through the hose 32 for inflating the bladder 24 to compress against the patient's limb 14 to make veins in the patient's limb 14 more visible. In this way a caregiver, such as a phlebotomist, can more easily identify and penetrate a vein for the purposes of phlebotomy.

The bulb 38 is comprised of a deformable material such as rubber or the like, and the bulb 38 has a primary end 40 and a secondary end 42. The primary end 40 is fluidly coupled to the second end 36 of the hose 32. The primary end 40 has a one way valve 44 that is integrated into the primary end 40 to pass air into the bulb 38 for inflating the bladder 24 while inhibiting air from passing out of the primary end 40 when the bulb 38 is compressed. Moreover, the bulb 38 may be an inflation bulb that is commonly employed on blood pressure cuffs, for example.

A valve 46 is provided and the valve 46 is fluidly integrated into the bulb 38. The valve 46 is positionable in an open condition to pass air outwardly from the valve 46 for deflating the bladder 24. Conversely, the valve 46 is positionable in a closed condition to inhibit air from passing outwardly from the valve 46 for retaining inflation of the bladder 24. The valve 46 is positioned at an intersection between the primary end of the bulb 38 and the second end 18 of the hose 32. Additionally, the valve 46 includes a knob 48 that is rotatably integrated into the valve 46 for positioning the valve 46 between the open condition and the closed condition. The valve 46 may be a two way air valve of any conventional design, such as is commonly employed on blood pressure cuffs or the like.

In use, the cuff 12 is wrapped around the patient's limb 14 such that the cuff 12 is strategically positioned with respect to a phlebotomy location on the patient's limb 14. The valve 46 is closed and the bulb 38 is repeatedly compressed to inflate the bladder 24. In this way the bladder 24 compresses against the patient's limb 14 in order to make veins in the patient's limb 14 more visible. Thus, the caregiver can more efficiently and precisely perform phlebotomy for medical purposes on the patient's limb 14. The valve 46 is opened to deflate the bladder 24 and the cuff 12 is removed from the patient's limb 14 when the phlebotomy is completed.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A phlebotomy cuff assembly for applying pressure on a patient's limb to make veins more visible for phlebotomy, said assembly comprising:
a cuff being configured to be wrapped around a patient's limb;
a bladder being integrated into said cuff;
a first mating member being coupled to said cuff;
a second mating member being coupled to said cuff, said second mating member being matable to said first mating member for forming said cuff into a closed loop wherein said cuff is configured to be retained around the patient's limb;
a hose being fluidly coupled to said bladder, said hose extending outwardly from said cuff;
a bulb being fluidly coupled to said hose wherein said bulb is configured to be compressed by a caregiver, said bulb urging air inwardly through said hose for inflating said bladder wherein said bladder is configured to compress against the patient's limb to make veins in the patient's limb more visible, said bulb being comprised of a deformable material; and
a valve being fluidly integrated into said bulb, said valve being positionable in an open condition wherein said valve is configured to pass air outwardly from said valve for deflating said bladder, said valve being positionable in a closed condition to inhibit air from passing outwardly from said valve for retaining inflation of said bladder.

2. The assembly according to claim 1, further comprising:
said cuff has a first end, a second end, a first surface extending between said first end and said second end, and a second surface extending between said first end and said second end, said cuff being elongated between said first end and said second end;
said bladder is positioned between said first surface and said second surface, said bladder being centrally positioned between said first end and said second end, said bladder being comprised of a fluid impermeable material wherein said bladder is configured to be inflated, said bladder having an inlet;
said first mating member is positioned on said first surface, said first mating member being positioned adjacent to said first end; and
said second mating member is positioned on said second surface, said second mating member being positioned adjacent to said second end.

3. The assembly according to claim 1, wherein:
said bladder has an inlet;
said hose has a first end and a second end, said first end of said hose being fluidly coupled to said inlet on said bladder;
said bulb has a primary end and a secondary end, said primary end being fluidly coupled to said second end of said hose, said primary end having a one way valve being integrated into said primary end wherein said one way valve is configured to pass air into said bulb for inflating said bladder; and
said valve is positioned at an intersection between said primary end of said bulb and said second end of said hose, said valve including a knob being rotatably integrated into said valve wherein said knob is configured to be gripped for positioning said valve between said open condition and said closed condition.

4. A phlebotomy cuff assembly for applying pressure on a patient's limb to make veins more visible for phlebotomy, said assembly comprising:
a cuff being configured to be wrapped around a patient's limb, said cuff having a first end, a second end, a first surface extending between said first end and said second end, and a second surface extending between said first end and said second end, said cuff being elongated between said first end and said second end;
a bladder being integrated into said cuff, said bladder being positioned between said first surface and said second surface, said bladder being centrally positioned between said first end and said second end, said bladder being comprised of a fluid impermeable material wherein said bladder is configured to be inflated, said bladder having an inlet;
a first mating member being coupled to said cuff, said first mating member being positioned on said first surface, said first mating member being positioned adjacent to said first end;
a second mating member being coupled to said cuff, said second mating member being matable to said first mating member for forming said cuff into a closed loop wherein said cuff is configured to be retained around the patient's limb, said second mating member being positioned on said second surface, said second mating member being positioned adjacent to said second end;
a hose being fluidly coupled to said bladder, said hose extending outwardly from said cuff, said hose having a first end and a second end, said first end of said hose being fluidly coupled to said inlet on said bladder;
a bulb being fluidly coupled to said hose wherein said bulb is configured to be compressed by a caregiver, said bulb urging air inwardly through said hose for inflating said bladder wherein said bladder is configured to compress against the patient's limb to make veins in the patient's limb more visible, said bulb being comprised of a deformable material, said bulb having a primary end and a secondary end, said primary end being fluidly coupled to said second end of said hose, said primary end having a one way valve being integrated into said primary end wherein said one way valve is configured to pass air into said bulb for inflating said bladder; and
a valve being fluidly integrated into said bulb, said valve being positionable in an open condition wherein said valve is configured to pass air outwardly from said valve for deflating said bladder, said valve being positionable in a closed condition to inhibit air from passing outwardly from said valve for retaining inflation of said bladder, said valve being positioned at an intersection between said primary end of said bulb and said second end of said hose, said valve including a knob being rotatably integrated into said valve wherein said knob is configured to be gripped for positioning said valve between said open condition and said closed condition.

5. A method of performing phlebotomy, the steps of the method comprising:
providing a cuff being wrappable around a patient's limb;
providing a bladder being integrated into said cuff;
providing a first mating member being coupled to said cuff;
providing a second mating member being coupled to said cuff;
providing a hose being fluidly coupled to said bladder;
providing a bulb being fluidly coupled to said hose;
providing a valve being fluidly integrated into said bulb;
wrapping said cuff around a patient's limb;
mating said first mating member to said second mating member to form said cuff into a closed loop around the patient's limb;
squeezing said bulb to inflate said bladder wherein said bladder is configured to compress against the patient's limb for making veins in the limb more visible;
performing phlebotomy on a vein;
opening said valve to deflate said bladder; and
removing said cuff from the patient's limb.
